(19) United States
(12) Patent Application Publication
(10) Pub. No.: US 2026/0196456 A1
(43) Pub. Date: Jul. 9, 2026

(54) METHOD AND DEVICE FOR RAPID DETECTION OF MICROPLASTICS AND NANOPLASTICS

(71) Applicant: NEW JERSEY INSTITUTE OF TECHNOLOGY, Newark, NJ (US)

(72) Inventors: Hao Chen, Livingston, NJ (US); Mengyuan Xiao, Newark, NJ (US)

(73) Assignee: NEW JERSEY INSTITUTE OF TECHNOLOGY, Newark, NJ (US)

(21) Appl. No.: 19/441,102

(22) Filed: Jan. 6, 2026

Related U.S. Application Data
(60) Provisional application No. 63/742,089, filed on Jan. 6, 2025.

(51) Int. Cl. H01J 49/04; G01N 1/40; G01N 33/44; H01J 49/00

(52) U.S. Cl. CPC H01J 49/0468; G01N 1/4077; G01N 33/442; H01J 49/004; G01N 2001/4088

(57) ABSTRACT

Disclosed are methods for direct, rapid and sensitive detection of plastic contaminants, such as microplastics (MPs) and nanoplastics (NPs), using flame ionization mass spectrometry (FI-MS) and flame-assisted atmospheric-pressure chemical ionization mass spectrometry (FAPCI-MS). Unlike traditional pyrolysis GC/MS methods, which require over 30 min for sample analysis and several hours for sample pre-treatment such as extraction, digestion and density separation, FI-MS allowed direct decomposition and ionization of MPs/NPs under atmospheric pressure in the front of a MS inlet, avoiding the use of a complicated pyrolysis system. The method achieves analysis of solid samples in under 10 seconds.

### RELATED APPLICATIONS

This application claims priority to and the benefit of U.S. Provisional Application No. 63/742,089, filed on Jan. 6, 2025, the entire contents of which is incorporated by reference herein.

### STATEMENT REGARDING FEDERALLY SPONSORED RESEARCH

This invention was made with government support under Agreement No. CHE-2203284 awarded by the National Science Foundation. The government has certain rights in the invention.

### FIELD OF THE DISCLOSURE

The present disclosure relates to a method for rapidly detecting plastics and other polymer contaminants in several types of media using flame ionization mass spectrometry (FI-MS) and flame-assisted atmospheric pressure chemical ionization mass spectrometry (FAPCI-MS).

### BACKGROUND

Microplastics (MPs, <5 mm in size) and nanoplastics (NPs, <1 μm in size) are emerging contaminants of environmental and human health concern. These tiny plastic particles (collectively MPs/NPs) originate from several sources, including the degradation of larger plastic debris,¹ microbeads in personal care products,² and synthetic fibers used in textile manufacturing. MPs/NPs pollute and persist in aquatic and terrestrial environments, posing numerous threats to ecosystems and human health.

Studies have detected MPs/NPs in drinking water, ambient air, human urine and blood, and in various sources of human food supply. Exposure to MPs/NPs can occur by ingestion, inhalation, and dermal contact, and MPs/NPs can accumulate in tissues, potentially causing inflammatory responses, oxidative stress, and other adverse health effects.^{3,4} Although the long-term effects of MP/NP exposure on human health are still being studied, growing evidence links MP/NP accumulation to various health issues, including but not limited to respiratory and gastrointestinal disorders, endocrine disruption, neurological disruption, and carcinogenicity.⁵

Studies have detected and quantified MPs/NPs in bottled water, suggesting that common consumable products distributed with plastic packaging can be contaminated with MPs/NPs on the shelf, and raising public concern over drinking water safety.⁶ Traditional methods for detecting the presence of MPs/NPs analysis include visual inspection under microscope, where plastic is visually identified based on particle shape and size. However, such labor-intensive methods are impractical for tracking the spread of MPs/NPs throughout the environment, which has escalated alongside plastic waste pollution associated with urbanization and industrial activity.

Advanced techniques have been developed with improved accuracy and efficiency for detecting MPs/NPs, including, but not limited to Fourier-transform infrared spectroscopy (FTIR),⁷ Raman spectroscopy, and micro-computed tomography (micro-CT).⁸ FTIR and Raman spectroscopy are nondestructive methods which analyze the unique spectral fingerprints of MPs/NPs. Polyethylene terephthalate (PET), a polyester commonly used in synthetic fabrics and single-use packaging plastics, contains repeating units of benzene rings and ester groups exhibiting aromatic C—H and ester carbonyl bond stretching vibrations at 3100-2800 cm⁻¹ and 1715 cm⁻¹, respectively, in FTIR spectra. These units also exhibit strong Raman scattering in Raman spectroscopy, with the carbonyl stretching peak at 1728 cm⁻¹ serving as the fingerprint marker for identifying PET. Micro-CT can distinguish microplastics from sediments based on material density, and generates high resolution three-dimensional images of MPs/NPs by directly scanning environmental samples.

However, existing advanced techniques each have shortcomings that limit their usefulness for detecting MPs/NPs in different contexts. For instance, micro-CT cannot effectively distinguish different types of plastics having similar densities. FTIR spectroscopy is highly effective for detecting plastic particles larger than 20 μm, but lacks the sensitivity to analyze nanoplastics or complex plastic mixtures due to overlapping absorption patterns. Raman spectroscopy of MPs/NPs has difficulties including background fluorescence interference, weak scattering signals, and long scan times for imaging large areas. Accordingly, there is a need to develop rapid, reliable methods for detecting, identifying, and quantifying MP/NP contaminants in various forms of media.

### SUMMARY

In accordance with embodiments of the present disclosure, methods are provided for direct, rapid and sensitive analysis of plastic contaminants, such as microplastics (MPs) and nanoplastics (NPs), using flame ionization mass spectrometry (FI-MS) and flame-assisted atmospheric-pressure chemical ionization mass spectrometry (FAPCI-MS).

In one embodiment, a method for rapid analysis of plastic contaminants could comprise positioning a sample comprising a plastic contaminant a first distance from an inlet of a mass spectrometer; heating the sample to an average temperature of from 200-2000° C. (e.g. in the presence of oxygen) to produce thermal decomposition products of the plastic contaminant; ionizing the thermal decomposition products; and detecting ionized thermal decomposition products with the mass spectrometer.

In accordance with embodiments of the present disclosure, the plastic contaminant could comprise a microplastic and/or nanoplastic.

In accordance with embodiments of the present disclosure, heating the sample could comprise applying a flame, electric heat source, or laser impact.

In accordance with embodiments of the present disclosure, heating the sample could comprise applying a flame directly or indirectly to the sample.

In accordance with embodiments of the present disclosure, the flame could be generated from a fuel source selected from propane, butane, alcohol, or the like.

In accordance with embodiments of the present disclosure, applying the flame could also ionize the thermal decomposition products.

In accordance with embodiments of the present disclosure, ionizing the thermal decomposition products could comprise using an ambient ionization method selected from flame ionization, atmospheric pressure chemical ionization, electrospray ionization, desorption electrospray ionization, sonic spray ionization, laser ionization, and plasma ionization.

In accordance with embodiments of the present disclosure, the method could further comprise preparing the sample from a medium containing a plastic contaminant.

In accordance with embodiments of the present disclosure, medium could be a powder, and preparing the sample could comprise heating a metal element to redness and contacting the powder with the heated metal element to prepare an immobilized powder sample on the metal element.

In accordance with embodiments of the present disclosure, the medium could be a liquid, and preparing the sample could comprise passing the liquid through a filter to collect the plastic contaminant on the filter and drying the filter to prepare a dry filtrate sample.

In accordance with embodiments of the present disclosure, the medium could be soil, and preparing the sample could comprise drying an amount of the soil to remove moisture and placing the soil in a vial having an opening.

In accordance with embodiments of the present disclosure, the medium could be a biological tissue, and preparing the sample could comprise homogenizing the biological tissue and depositing an amount of the homogenized tissue on a substrate.

In accordance with embodiments of the present disclosure, the method could be completed in 10 seconds or less.

In accordance with embodiments of the present disclosure, the sample can be on a vial, substrate, or support, and heating the sample could comprise heating the sample indirectly by heating the vial, substrate or support.

In accordance with embodiments of the present disclosure, ionizing the thermal decomposition products could comprise applying an ambient ionization method above the sample while heating the sample indirectly, and the ambient ionization method can be selected from flame ionization, atmospheric pressure chemical ionization, electrospray ionization, desorption electrospray ionization, sonic spray ionization, laser ionization, and plasma ionization

In accordance with embodiments of the present disclosure, ionizing the thermal decomposition products could comprise applying a propane flame having an average temperature of from 500-2000° C. above the sample while heating the sample indirectly.

In accordance with embodiments of the present disclosure, ionizing the thermal decomposition products could comprise applying a voltage in a range of from 1 to 5.0 kV to a metal needle placed above the sample while heating the sample indirectly.

In accordance with embodiments of the present disclosure, the vial, substrate or support could comprise glass, glass fiber, or metal.

In accordance with embodiments of the present disclosure, the ionized thermal decomposition products could comprise one or more characteristic ions of the plastic contaminant.

In accordance with embodiments of the present disclosure, the plastic contaminant could comprise polyethylene terephthalate (PET), and the characteristic ions could comprise one or more of vinyl terephthalate cation (m/z about 193.05) and 4-acetylbenzaldehyde cation (m/z about 149.06) with the mass spectrometer in positive ion mode, or vinyl terephthalate anion (m/z about 191.03) with the mass spectrometer in negative ion mode.

In accordance with embodiments of the present disclosure, the plastic contaminant could comprise polystyrene (PS), and the characteristic ions comprise one or more of 1,3-diphenylpropene cation (m/z about 195.116) and but-3-ene-1,3-diphenylpropene cation (m/z about 207.116) with the mass spectrometer in positive ion mode.

In accordance with embodiments of the present disclosure, the method could further comprise quantifying an amount of the plastic contaminant present in the sample by analyzing a signal intensity of a detected ionized thermal decomposition product. In one or more embodiments, the signal intensity is compared against calibration curves of standards.

In accordance with embodiments of the present disclosure, the method could further comprise analyzing one or more detected ionized thermal decomposition products using tandem mass spectrometry (MS/MS).

Other steps and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in using the disclosed method for rapid detection of microplastics and nanoplastics and associated systems and methods, reference is made to the accompanying figures, wherein:

FIG. 1 is a schematic showing a proposed thermal decomposition pathway of polyethylene terephthalate (PET).

FIG. 2A is a schematic depicting flame ionization mass spectrometry (FI-MS) analysis by direct combustion of a solid sample in accordance with embodiments of the present disclosure.

FIG. 2B is a schematic depicting FI-MS analysis of a powder sample by immobilization and combustion in accordance with embodiments of the present disclosure.

FIG. 2C is a schematic depicting FI-MS analysis of a liquid sample by filtration and combustion of filtered contaminants in accordance with embodiments of the present disclosure.

FIG. 2D is a schematic depicting FI-MS analysis of a solid environmental sample by indirect heating and flame ionization of vaporized contaminants in accordance with embodiments of the present disclosure.

FIG. 3 shows spectra from FI-MS analysis of standard PET microplastic powder (top); bulk plastic from a commercially available clear plastic water bottle (middle); and bulk plastic from a commercially available green plastic water bottle (bottom) in accordance with embodiments of the present disclosure.

FIG. 4 shows a specFtrum from tandem mass spectrometry (MS/MS) of the protonated vinyl terephthalate fragment (m/z 193.05) generated from flame ionization of PET in accordance with embodiments of the present disclosure.

FIG. 5 shows spectra from FI-MS analysis of commercially available bottled water passed through cellulose-based filter paper (top) and control blank filter paper (bottom) in accordance with embodiments of the present disclosure.

FIG. 6 shows spectra (left) and calibration curve (right) from FI-MS analysis of variable concentration samples of standard PET microplastic powder in solid dilution with deactivated silica in accordance with embodiments of the present disclosure.

FIG. 7 shows a spectrum (top) and calibration curve (bottom) from FI-MS analysis of variable concentration samples of standard polystyrene (PS) nanoplastic powder diluted in deionized water in accordance with embodiments of the present disclosure.

FIG. 8 shows spectra collected from FI-MS analyses of soil samples unspiked (top) and spiked (bottom) with PET microplastics in accordance with embodiments of the present disclosure.

FIG. 9 is a schematic showing a method for detecting MPs/NPs using flame assisted atmospheric-pressure chemical ionization mass spectrometry (FAPCI-MS) in accordance with embodiments of the present disclosure.

FIG. 10 shows spectra obtained from FAPCI-MS (top) and FI-MS (middle) analysis of PS nanoplastics in accordance with embodiments of the present disclosure, and a simulated signal from a PS dimer ion (bottom).

FIG. 11 shows spectra (left) and calibration curve (right) obtained from FAPCI-MS analysis of PS-spiked and control samples of pork exudate in accordance with embodiments of the present disclosure.

FIG. 12 shows spectra (left) and calibration curve (right) obtained from FAPCI-MS analysis of PS-spiked and control samples of homogenized salmon in accordance with embodiments of the present disclosure.

FIG. 13 shows spectra obtained from FAPCI-MS analysis of PVC-spiked sand in accordance with embodiments of the present disclosure (top left), with MS/MS analysis (top right) and signal intensity of the major PVC decomposition product (1-methylnaphthalene) from the spiked (bottom left) and control samples (bottom right).

FIG. 14 shows a spectrum obtained from FAPCI-MS analysis of standard PET MPs in accordance with embodiments of the present disclosure.

FIG. 15A shows spectra obtained from FAPCI-MS analysis and triplicate analysis of the vinyl terephthalate ion from bottled water before and after being microwave heated, in accordance with embodiments of the present disclosure.

FIG. 15B shows spectra obtained from FAPCI-MS analysis and triplicate analysis of the vinyl terephthalate ion from the filter paper used to filter bottled water before and after being microwave heated, in accordance with embodiments of the present disclosure.

### DETAILED DESCRIPTION

Embodiments of the present disclosure include systems and methods for detecting microplastics and nanoplastics (MPs/NPs) using flame ionization mass spectrometry and flame-assisted mass spectrometry. Detectable polymer and plastic species in MPs/NPs include polyethylene terephthalate (PET), polystyrene (PS) and polyvinyl chloride (PVC), although it should be understood that the systems and methods disclosed herein could generally be applied to other microplastics and nanoplastics species, or generally applied to other plastics and polymers.

There is a growing need to monitor the infiltration of potentially harmful MPs/NPs into these and other materials and environmental destinations at a large scale. Important performance factors for techniques for assessing MP/NP contamination include, but are not limited to: sensitivity (ability to detect a plastic contaminant in trace or low amounts or concentrations), accuracy (ability to correctly quantify, identify, and/or distinguish amongst closely related contaminant species), reliability (technique is repeatable; instrumentation is not prone to malfunction or compromise), scalability (ability to accommodate increased demand), speed (time required to provide desired analysis, including sample procurement and processing), versatility (ability to analyze a broad range of plastic contaminant species and properties thereof; ability to analyze multiple sample forms and samples comprising multiple materials and substances), ease of use (performable with minimal training and low risk of failure), and affordability.

Every analytical method is limited as to which chemical properties it can measure, and every method has process requirements pertaining to how a sample is prepared and analyzed. Together, these and other practical considerations can impact method performance along the factors discussed above in ways that may limit a method's usefulness for a given application. Analytical methods for identifying chemical compositions have been used to ascertain contaminants present in bulk substances of interest relative to environmental and human health concerns. These methods, including advanced spectroscopic methods discussed herein, can be used for detecting and identifying MPs/NPs with great precision and sensitivity, and have been adapted to assess MPs/NPs present in biological samples such as plant and animal tissues, environmental samples such as water and soil, and in food and beverages consumed by humans. However, currently available methods suffer from several shortcomings that limit their suitability to be used for large scale monitoring and/or screening of MP/NP contamination in diverse forms of media, notably including, but not limited to, limited versatility of species that can be analyzed, labor and time intensive sample preparation requirements, long analysis times, low precision, and high operation costs.

Mass spectrometry (MS) is an established powerful technique for chemical analysis. Compared to spectroscopic methods, MS is highly sensitive and provides molecular weight information for accurate chemical identification. As with spectroscopic methods, however, challenges also arise in the use of MS for MP/NP analysis. For example, due to their strong hydrophobic nature and large molecular weight, synthetic plastic compounds are insoluble in water and most organic solvents typically used to prepare samples compatible with MS instruments, which make it difficult to analyze MPs/NPs directly by MS. One approach to the challenge of sample preparation for MS has been solution has been to degrade polymers into small volatile molecules via thermal decomposition as part of a technique called pyrolysis-gas chromatography-mass spectrometry (pyr-GC/MS). Pyr-GC/MS is a powerful technique used to identify and quantify MPs/NPs in which the sample for analysis is first subjected to pyrolysis before being carried into the gas chromatography (GC) column for separation and detection by MS.¹⁰

Pyr-GC/MS can detect thermal decomposition products from the pyrolysis of PET. Scheme 1 illustrates a suggested pathway for the pyrolysis of PET leading to products terephthalic acid and 4-((vinyloxy) carbonyl)benzoic acid. However, pyr-GC/MS requires a complex pyrolysis chamber and tedious sample preparation process prior to GC/MS analysis.¹⁰ In some cases, pyr-GC/MS can require nitric acid digestion of the plant contained in samples, density separation, and multiple organic extractions before introducing the sample to the pyrolysis chamber. Accordingly, pyr-GC/MS analysis of MPs/NPs can require hours to prepare the sample and an additional 30 minutes or more to conduct the GC/MS analysis. Therefore, an improved fast, direct and sensitive method is needed for detecting and quantifying MPs/NPs in various forms of environmental and commercial materials.

Flame ionization (FI) is commonly used in gas chromatography (GC) to detect organic compounds^{11, 12} by converting them into ions through thermal decomposition in a flame. Using ambient flame ionization mass spectrometry (AFI-MS), a butane flame enables ionization via protonation, allowing for rapid and sensitive detection of organic samples. For example, sodium saccharin can be quickly detected and quantified by this method. It will be understood that other flames and temperatures can be used, such as, for example, in a range of from 200-2000° C.

Disclosed are systems and methods for rapid and direct detection of MPs and NPs from various sample matrices (e.g., water, soil, food, beverages, blood, biological tissue, etc.) using high resolution mass spectrometry. Samples containing MPs and/or NPs are heated by a direct flame to generate decomposition vapors. The resulting decomposition vapors are then ionized by ambient ionization to generate characteristic ions of MPs and NPs in the sample for mass spectrometric (MS) detection and identification. In embodiments of the disclosure, ambient ionization methods may include, but are not limited to, flame ionization, atmospheric pressure chemical ionization, electrospray ionization, desorption electrospray ionization, sonic spray ionization, laser ionization, plasma ionization, and other ambient ionization methods based on impact of high energy particles, laser or plasma. Due to the minimal sample preparation required by methods of the disclosure, methods may be performed in mere seconds. Methods of the disclosure may be used for the detection, identification, and quantification of MPs and/or NPs in various sample formats.

Aside from the various illustrative embodiments described herein, other embodiments are contemplated and are capable of being practiced or being carried out by other ways than those described herein. Thus, it is to be understood that the present disclosure is not limited in its application to the details of construction and the arrangements of components set forth in the following description or illustrated in the drawings.

### Definitions

As used herein, the term “plastic” refers to synthetic or semi-synthetic polymers which can be shaped or molded when soft and then hardened to retain the given shape. Examples of plastics include, but are not limited to, polyethylene (PE), polypropylene (PP), polystyrene (PS), and polyethylene terephthalate (PET). The terms “microplastics” (MP) and “nanoplastics” refers to plastic particles having particle sizes less than 5 μm or 1 μm, respectively.

As used herein, the term “ionization” refers to a means of converting a chemical compound into an ion, radical, or other electrically charged species of said compound or fragment thereof, for intake and analysis by a mass spectrometer.

In exemplary embodiments, plastics (e.g. MP/NP particles) are decomposed by a heat source (e.g. flame) to produce thermal decomposition products, which can be further ionized (e.g. by a flame or ionization probe) for MS detection. Disclosed is a fast, direct, sensitive and convenient method to detect and quantify MPs and NPs using FI-MS. For instance, by exposing PET-containing samples to a direct flame near a mass spectrometer inlet, flame-ionized decomposition products of PET can be detected by MS.

Although specific compounds and materials are discussed with regard to exemplary embodiments of the present disclosure, it is understood that other suitable compounds and materials can be used. Similar quantities or measurements may be substituted without altering the method embodied below.

Materials and instruments used in experiments conducted using methods of the present disclosure are discussed herein. Bottled water was purchased from a large local retailer. Standard 300 μm microplastic PET powder was purchased from Goodfellow Corporation (Boulder City, Nevada). Polystyrene (PS) nanoplastics latex bead solutions (70% water; 30% by weight; average particle size 100 nm) and cellulose membrane filters (0.7 μm Pore Size, 47 mm diameter) were purchased from Millipore Sigma (Temecula, California). Bic multi-purpose butane lighters (average flame temperature 600-800° C.) were used as a source of direct flame and to perform other heating.¹³ A propane torch (Bernzomatic; average flame temperature 700-1600° C.).14 Vacuum filter apparatus was purchased from ChemGlass Life Science CG-1424 (Vineland, NJ). Deionized water was generated using Direct-Q 5UV (Millipore Sigma). Experiments of the present disclosure were performed using a high-resolution Orbitrap Q Exactive mass spectrometer (Thermo Scientific, San Jose, CA). The MS instrument was set at 140,000 resolution, positive ion mode, 3 microsecond per scan, Automatic Gain Control (AGC) as 1E6, and maximum inject time as 30 microseconds. Tandem mass spectrometry (MS/MS) was performed using all ion fragmentation mode with HCD energy as 50 eV and the fragmentation width was set to 0.4 m/z.

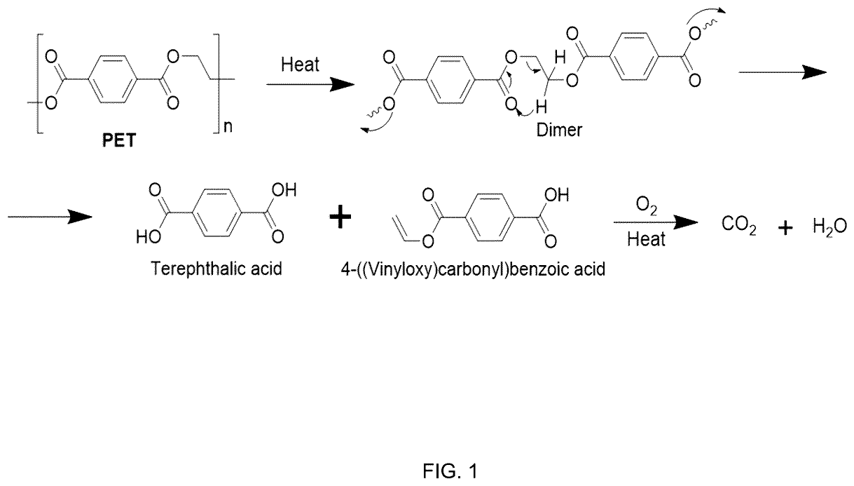

FIG. 1 is a schematic showing a proposed reaction mechanism for the thermal decomposition of PET yielding decomposition products that have been observed by MS analysis. Application of heat to the PET polymer yields fragment molecules, such as terephthalic acid and 4-((Vinyloxy) carbonyl)benzoic acid, that can be then be ionized by a flame for direct analysis by MS. Competition amongst processes that may occur upon application of flame to analyte chemicals may be modulated by adjustment of variables including, but not limited to the temperature of the flame, fuel flow rate, distance of the flame from the sample, distance of the sample from the MS inlet, ambient oxygen, and composition of the sample containing the analyte chemical. Thermal decomposition and ionization pathways may be aggressive, resulting in fragmentation (i.e., severing and/or rearrangement of covalent bonds in analyte molecules to yield smaller “fragment” ions), or gentle, wherein an intact analyte molecule is ionized by protonation or other means with little to no fragmentation. Without intending to be bound by any theory, gentle ionization may not be appropriate for FI-MS analysis of polymer species, as commonly found in MPs/NPs, due to large molecular weights associated with repeated monomer base units that may be incompatible with MS analysis if left intact and ionized by simple protonation. Hence, thermal decomposition, as by the proposed pathway for PET shown in FIG. 1, is an important process preceding ionization for MS analysis of some embodiments of methods of the present disclosure.

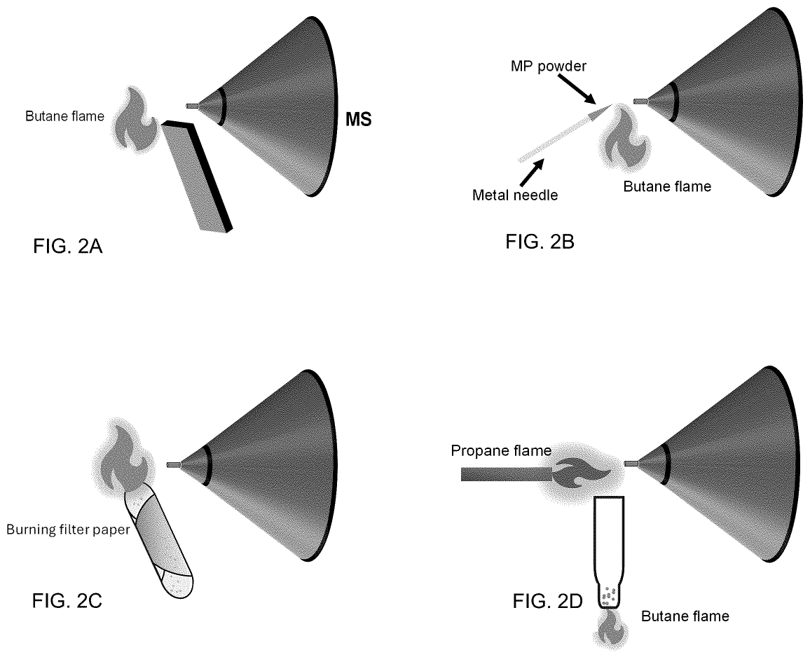

FIGS. 2A-D are schematics showing non-limiting examples of methods of FI-MS analysis in accordance with the present disclosure. These examples are further discussed relative to their use in experiments described herein. FIG. 2A depicts an exemplary method for analyzing MPs/NPs present in solid materials, such as bulk plastic packaging, by application of a direct flame to a solid sample near the inlet of a mass spectrometer. FIG. 2B depicts combustion of MP/NP powder adsorbed on a metal rod. FIG. 2C depicts combustion of a cellulose-based filter paper carrying MPs/NPs; for example, liquids containing MPs/NPs or other polymer contaminants may be passed through a filter which is then ignited by a direct flame in the vicinity of a MS inlet. FIG. 2D depicts FI-MS analysis of other sample types, such as, in certain non-limiting examples, solid composites, bulk environmental materials such as soil, or other solid or liquid mixtures containing MPs/NPs or other polymer contaminants. In the scheme of FIG. 2D, MPs/NPs-containing soil samples can be placed in an inert vial and subjected to dual-flame heating. Without intending to be bound by any theory or suggested mechanism, heating the sample indirectly through the vial by a first flame or other heat source may decompose the sample, cause desorption and vaporization of MPs/NPs contained therein, while a flame applied directly above the vial and near the MS inlet can be used to ionize gas-phase compounds released from the sample as they emerge from the vial. In some embodiments, the indirect heat can be supplied by a butane flame, while the direct heat can by supplied by higher-temperature flame such as propane to induce ionization of compounds released from the sample by the indirect heat. The direct flame and indirect flame may, in some embodiments, be generated from a fuel source such as propane, butane, alcohol, or the like.

Embodiments according to the present disclosure have been performed using a high-resolution mass spectrometer. In some embodiments, the commercial ion source of the mass spectrometer can be removed, leaving the mass inlet of the spectrometer exposed to air. In an embodiment according to the scheme of FIG. 2A, a single-use plastic water bottle was cut into rectangular strips (2 cm×3 cm) and a strip positioned in front of a mass inlet using a tweezer. The plastic strip was heated with a direct flame from a Bic multi-purpose butane lighter having a flame temperature between 600-800° C. Without intending to be bound by theory, in this embodiment, it is expected that exposure to the direct flame in ambient air results in both the thermal decomposition of plastic compounds and the ionization of the resulting thermal decomposition products in the same flame. Thus, this method can provide ionized fragments of polymers for detection by MS analysis without cumbersome sample preparation or expensive processing equipment.

Multiple sample types can be analyzed by embodiments of the method of the present disclosure. In one embodiment, a stainless-steel needle was first heated with a butane lighter to redness. The hot needle tip was placed directly into micro plastic powders to immobilize MP/NP powder on the needle surface, as depicted in the scheme of FIG. 2B. In this example, the needle bearing immobilized MP/NP powder was then stabilized in front of the MS mass inlet and heated by a Bic Multi-purpose butane lighter having a flame temperature between 600-800° C. In embodiments of the present disclosure, MS data is collected continuously during application of direct heat to the sample.

According to a further embodiment, two liters of purified bottled drinking water were filtered through a cellulose-based fiber filter paper (0.7 μm pore size) using a vacuum filtration system. Glassware was thoroughly rinsed and cleaned with a 50:50 water/methanol solution and subsequently dried in an oven prior to use. After the filtration, the filter paper was dried to remove residual moisture before analysis. Drying can be performed using, e.g., an oven, a hotplate, or other suitable means for drying without combusting the filter. The dried filter paper was then rolled into a cylinder shape and positioned near the MS mass inlet, as shown in the scheme depicted in FIG. 2C. The paper was then ignited using a Bic multi-purpose butane lighter having a flame temperature of from 600-800° C. and MS data was collected concurrently.

According to further embodiments, samples were prepared for FI-MS analysis by collecting local soil. Soil samples included as-found soil samples and soil samples ‘spiked’ MPs or NPs. Analyses were conducted by a method as depicted by the scheme of FIG. 2D. Soil samples were placed in small glass vials (e.g., 5 mm ID×29 mm height), which was fastened close to the mass spectrometer inlet. A flame from a Bic multi-purpose butane lighter having a flame temperature of from 600-800° C. was applied to the sample indirectly, for example by positioning the flame a distance from bottom of the glass vial containing the soil sample. In some embodiments, the flame is applied to contact the vial containing the sample. In non-limiting embodiments, heat applied to a sample contained in a vial can cause the sample to do one or more of decompose the sample (e.g. the soil) and cause the desorption or vaporization of MPs/NPs and/or other contaminant species from the sample, causing them to exit the vial. Without intending to be bound by theory, it is expected that vapor exiting the sample can comprise thermal decomposition products of MPs/NPs from the soil sample. A propane torch was used to apply a flame having an average temperature of from 700-1600° C. at, near, or adjacent the opening of the vial to ionize decomposition products contained in the vapor prior to intake by the MS mass inlet, as depicted in FIG. 2D. It will be understood that flames can be used having different average temperatures, such as 500-2000° C., for example.

Embodiments according to the present disclosure were used to conduct quantitative analyses of MPs/NPs present in bulk sample materials. In a non-limiting example, a PET standard was prepared through solid dilution in deactivated silica powder to ensure a uniform matrix. For example, to prepare a sample for FI-MS analysis containing 1 μg of PET microplastics, 1 mg of PET standard microplastic powder (300 μm average particle size) was thoroughly mixed with 999 mg of silica powder using a vortex mixer and 1 mg of the resulting mixture was placed in a glass vial. 1 mg samples containing 0, 0.5 μg, 5 μg, 15 μg, and 20 μg, respectively, of PET microplastic in silica were prepared in a similar manner and measured three times by FI-MS by the method depicted in FIG. 2D.

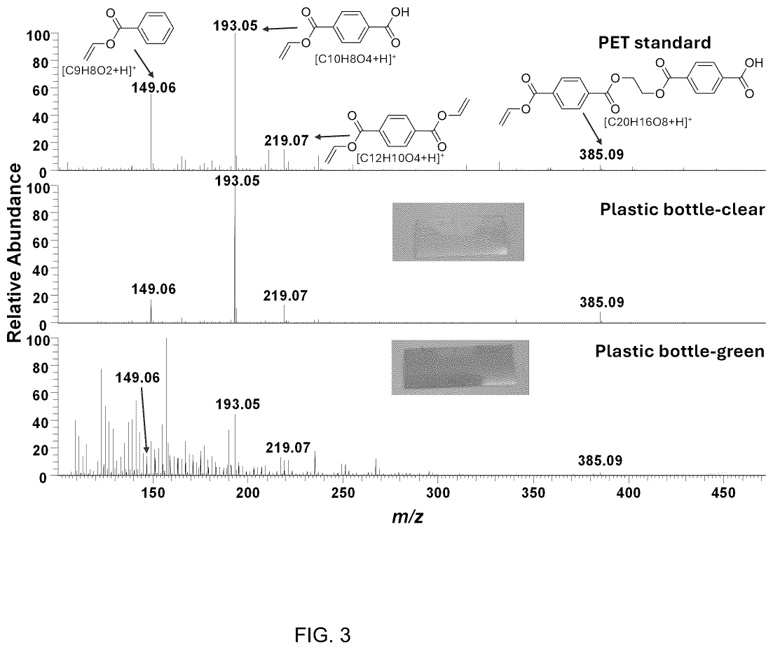

FIG. 3 (top) illustrates a MS spectrum obtained from FI-MS analysis PET standards microplastic powder (size: 300 μm). The collected data enabled identification of various chemical species associated with thermal decomposition of the PET polymer, including, for example, vinyl terephthalate (m/z 193.05), 4-acetylbenzaldehyde (m/z 149.06), terephthalic acid bimolecular (m/z 385.09), and di-vinyl terephthalate (m/z 219.07). These compounds have been previously detected by photo-ionization following PET pyrolysis,¹⁰ with the observed m/z values corresponding to fragments included in proposed decomposition pathways (see FIG. 1). Observation of these characteristic decomposition products demonstrates that FI-MS can be used to ionize and detect PET MPs.

FI-MS was further employed to analyze two different commercially available single-use plastic water bottles. FIG. 3 (middle) shows a MS spectrum collected from FI-MS analysis of a sample from a clear plastic bottle subjected to a direct propane flame. This spectrum shows peaks consistent with the PET standard, indicating that the bottle comprised bulk PET with minimal significant additives. FIG. 3 (bottom) shows a MS spectrum collected from FI-MIS analysis of a green plastic bottle. This spectrum shows increased background noise compared to the spectrum obtained from analysis of the clear plastic bottle. Still, the presence of four characteristic ions at m/z 149.06, m/z 193.05, m/z 219.07 and m/z 385.09 was confirmed, demonstrating that methods of the present disclosure can effectively identify PET even in samples in which PET is mixed with dyes and other additives.

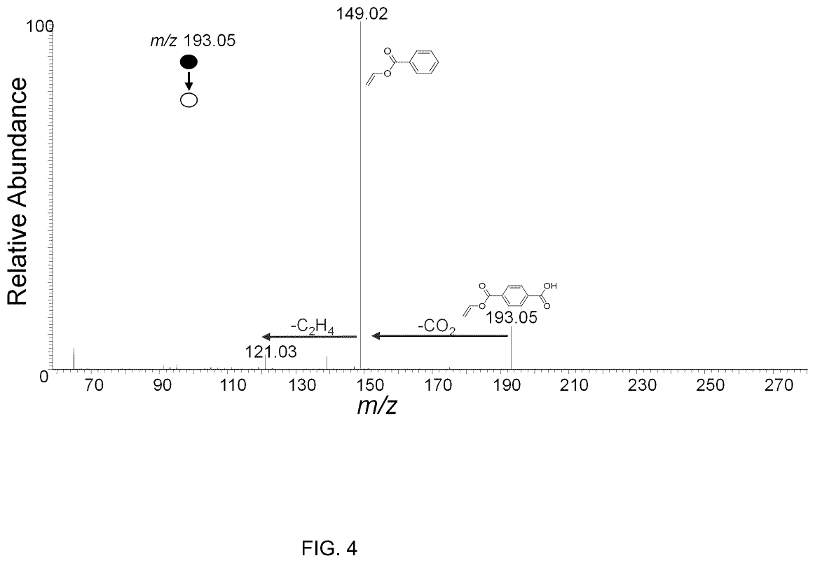

Significantly, the total time for analysis of each sample of FIG. 3 was just 10 seconds, compared to other methods such as pyr-GC/MS which take more than 30 min for analysis after several additional hours of arduous sample preparation. To confirm the structure of PET thermal decomposition products, tandem mass spectrometry (MS/MS) was performed for the protonated vinyl terephthalate (m/z 193.05). Higher-energy collisional dissociation (HCD) with 50 eV voltage was used for fragmentation of the vinyl terephthalate ion. A resulting spectrum is shown in FIG. 4, showing that fragment ions m/z 149.02, 165.07 and 121.03 were produced, probably due to consecutive losses of CO₂ and C₂H₄, consistent with a fragmentation pattern of vinyl terephthalate.

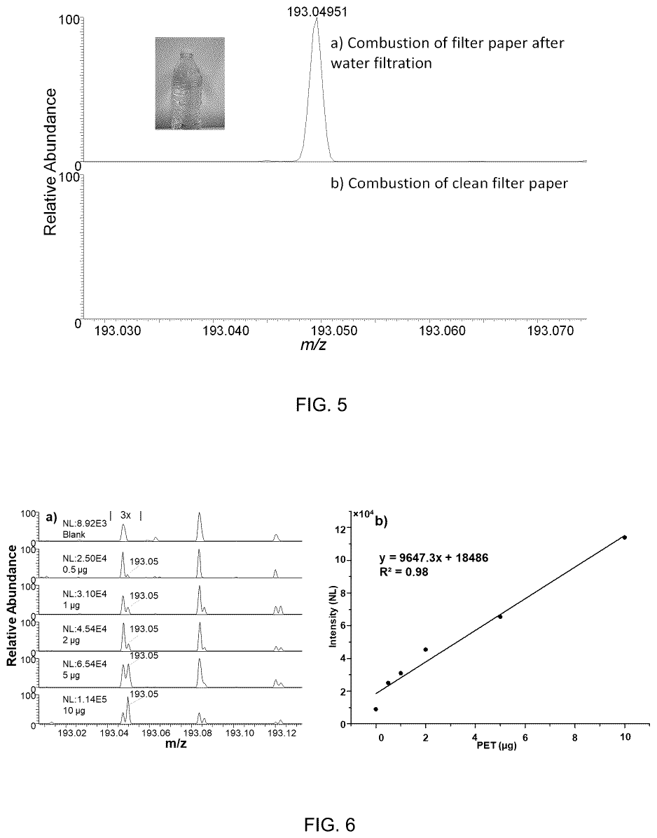

FIG. 5 demonstrates detection by FI-MS of PET microplastics in commercial purified bottled water obtained from local retail stores. Clean filter paper was first analyzed in a control experiment in which the paper was ignited by a direct butane flame in front of the MS inlet with continuous flame application throughout combustion. With three repeated tests, no m/z 193.05 signal was observed, confirming that the clean filter paper was free from microplastic contamination. Subsequently, water from two bottled samples (1 L) was filtered through another piece of clean filter paper using a vacuum filtration system and dried as described herein. Combustion of the dried filter paper in front of the MS inlet performed in same manner yielded a m/z 193.05 signal with a measured mass error of only 1 ppm. These results confirm that MPs/NPs from bottled water can be successfully detected by FI-MS analysis after quick and simple filtration in accordance with embodiments of the present disclosure. Whereas previous studies reporting using 20 L of bottled water¹⁵ for MP and NP analysis, only two bottles (1 L) of water were used in experiments described herein, evidencing the high sensitivity of disclosed methods for MPs/NPs in water samples.

Methods of FI-MS of the present disclosure may be used for quantitative analysis of microplastics and nanoplastics. Experiments were conducted in which powder samples containing, respectively, 0, 0.5 μg, 1.0 μg, 2 μg, 5 μg, and 10 μg of standard PET microplastic powder (particle size: 300 μm) were prepared by solid dilution in deactivated silica powder as discussed herein and analyzed three times by FI-MS with indirect heating as set forth herein and shown in FIG. 2D. FIG. 6 shows that the characteristic PET peak of m/z of 193.05 demonstrates intensity increasing as the amount of PET in the dilution sample increased. A linear calibration curve was obtained with equation y=9647.3x+18486 (R²=0.98). The limit of detection (LOD) of PET microplastics was calculated to be 0.25 μg by classical criteria:

$$LOD = \frac{3 \times SD}{S}$$

where SD is the standard deviation of the blank (accounting for noise) and S is the slope of the calibration curve from the standards.

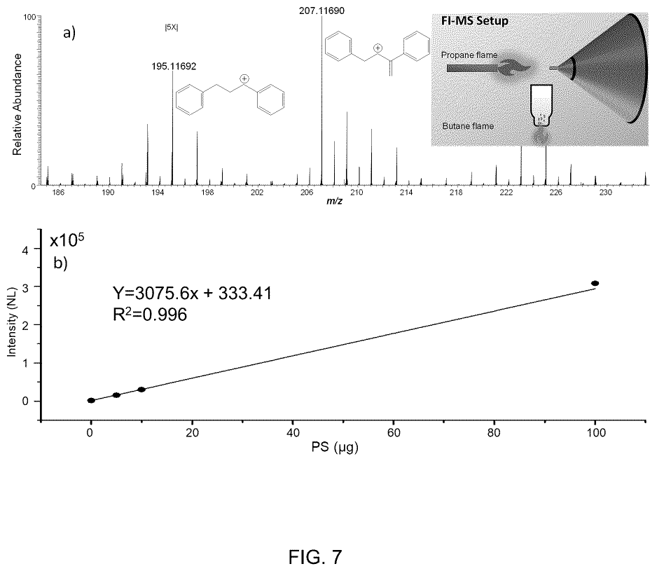

In another quantitative analysis experiment, samples were prepared by diluting standard PS nanoplastic latex bead solution (100 nm average particle size) in deionized water and analyzed by FI-MS using the method of FIG. 2D. Different amounts of standard 100 nm PS nanoplastics were measured. FIG. 7 (top) shows detection of ions of PS decomposition products such as 1,3-diphenylpropene (measured m/z 195.11692, theoretical m/z 195.11683) and but-3-ene-1,3-diphenylpropene (measured m/z 207.11691, theoretical m/z 207.11683), with mass errors of 0.5 and 0.44 ppm, respectively. These observed decomposition products matched previously reported results from pyrolysis GC-MS studies, 16, 17 and a calibration curve with strong linearity (R²=0.996, FIG. 7 (bottom) was obtained. In addition, the LOD for PS nanoplastics detection was calculated to be 0.7 μg, demonstrating the high sensitivity methods of the present disclosure for MP/NP analyses. These results demonstrate the ability of methods of the present disclosure to accurately quantify microplastics and nanoplastics in sub-microgram scales.

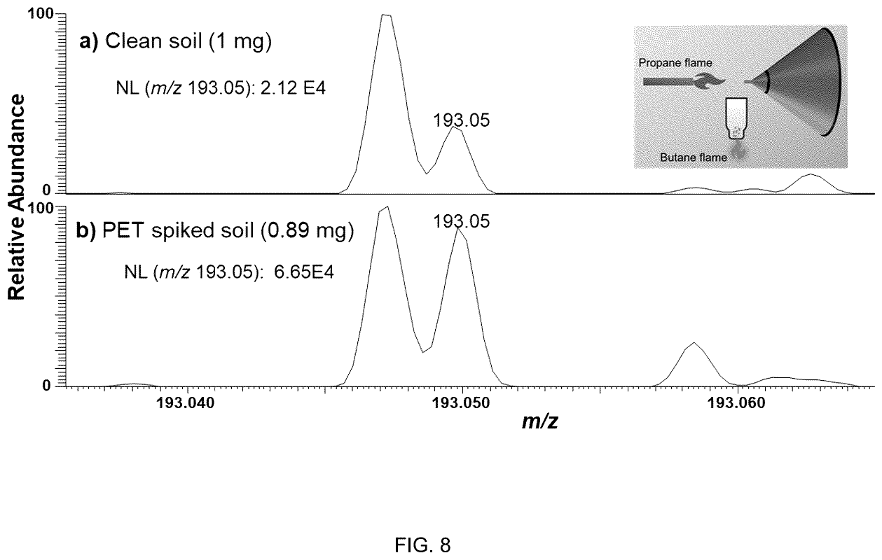

To detect PET microplastics in soil, soil samples were collected from the local neighborhood and dried at 80° C. to remove moisture. Dried samples were then placed in a glass vial, which was positioned in front of the MS inlet, following the same procedure as used for the FI-MS of PET standard microplastics (FIG. 2D). An unspiked 1 mg soil sample was first analyzed with FI-MS. The decomposed PET product ions of m/z 149.06, 193.05, and 219.07 from this soil sample were detected with weak intensity, indicating that the soil contained PET (FIG. 8, top). From the acquired MS spectrum of this soil sample (FIG. 8, top), the decomposed PET product ion of 193.05 was detected with weak intensity (2.12E4, blank sample in FIG. 6, left). To distinguish whether the m/z 193.05 signal was produced by PET contamination or by background, MS/MS of the m/z 193.05 was conducted but its characteristic fragment ion at m/z 149.02 was not detected, suggesting that the m/z 193.05 signal from the soil was not from PET and the soil we collected was free from PET.

To further test the feasibility of identifying and quantifying PET in the soil sample, a 500 mg soil was spiked with 3 mg of standard PET microplastics (average size: 300 μm), creating a 6000 ppm PET microplastics-containing soil sample. A 0.89 mg of the spiked sample was tested with FI-MS. The signal intensity of PET-specific peak at m/z of 193.05 (FIG. 8, bottom) was 2.82E5 for this PET-spiked soil sample. Using the calibration curve in FIG. 6, the mass of PET detected in this PET-spiked soil was quantified to be 4.98 μg (5595 ppm). Comparing this to the theoretical value of 6000 ppm, the quantitation exhibited only a 6.8% deviation from theory. This result emphasizes that the present FI-MS can not only identify PET microplastics from soil but could accurately quantify it.

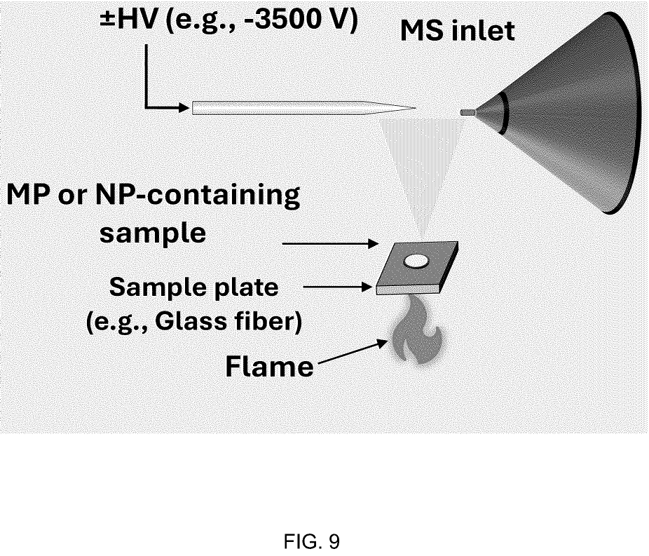

In a further embodiment shown in FIG. 9, a flame ionization mass spectrometry (FI-MS) system was adapted to create a flame-assisted atmospheric-pressure chemical ionization mass spectrometry (FAPCI-MS) system, which exhibited improved sensitivity and reproducibility for detecting micro- and nanoplastics (MP/NPs) in low abundance in biological samples. Methods of the present disclosure used a corona discharge generated from the tip of a needle charged with a high voltage near the MS inlet to ionize analyte vapors released by heating the MP/NP-containing sample using a lighter (e.g., a Scripto HW12 Utility Lighter). In some embodiments, the high voltage can be a voltage such as ±1 kV, or 2 kV, or 3 kV, or 4 kV, or 5 kV, or 6 kV. In certain embodiments of FI-MS analysis, FI-generated ion signal stability in MS spectra can exhibit disturbances, potentially caused by flame fluctuations caused by ambient air turbulence around the MS inlet. A fixed position of the ionization needle in FAPCI-MS as depicted in the scheme shown in FIG. 9 can provide a more stable signal. In experiments discussed herein, the ionization needle was positioned a distance from the MS inlet, with a sample plate (glass fiber) placed a distance below the needle tip, to reduce distance between the analyte and the corona discharge region and thereby improve sensitivity of the analysis. In some embodiments, the distance from the MS inlet can be a distance such as 5 mm, or 8 mm, or 10 mm, or 11 mm, or 12 mm, or 13 mm, or 14 mm, or 15 mm, or 20 mm, or 25 mm, or 30 mm, or any range therebetween. In some embodiments, the distance of the class fiber below the needle tip can be a distance such as 0.5 mm, or 1 mm, or 1.5 mm, or 2 mm, or 2.5 mm, or 3 mm, or 5 mm, or 10 mm.

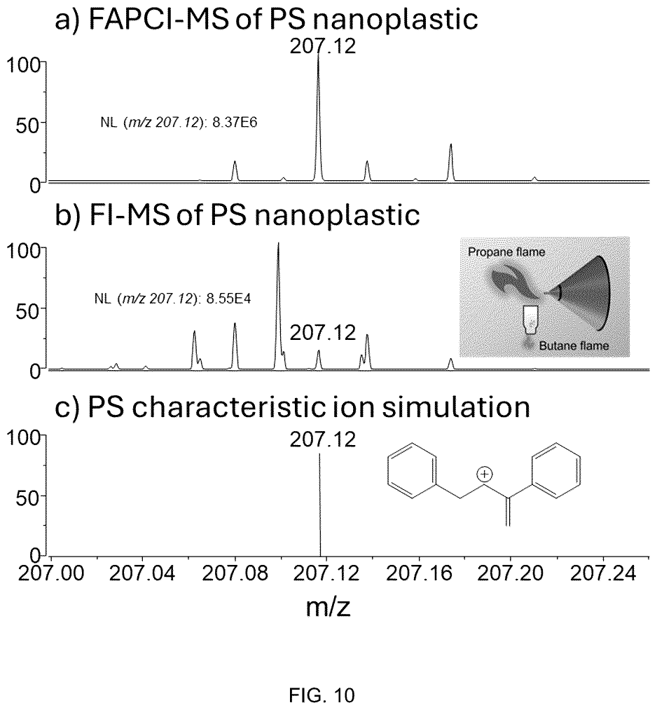

Some embodiments of the present disclosure require as little as 1 μL of homogenized sample deposited on the glass fiber for analysis, greatly reduced the sample amount required for traditional methods (e.g., pyrolysis GC-MS). As shown in the spectra of FIG. 10, analysis by FAPCI-MS can increase the signal intensity (m/z 207.12) of standard PS by approximately 100-fold (8.37E6, top) compared with the FI-MS analysis (8.55E4, middle).

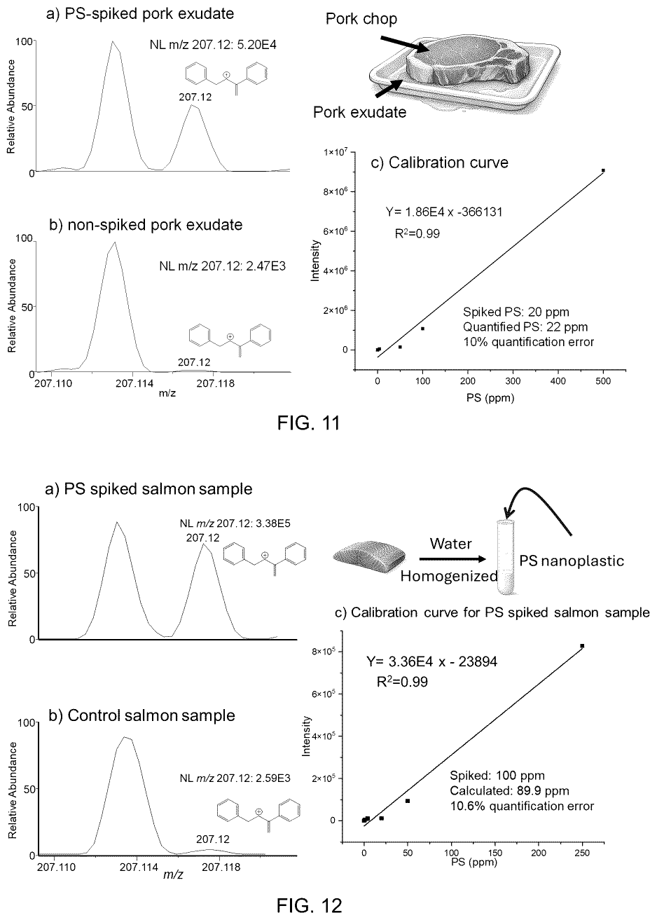

Further embodiments of the present disclosure can provide high sensitivity and accuracy for detecting PS in spiked pork exudate samples, suggesting the applicability of embodiments for screening real-world biological samples and environmental samples for MPs/NPs contamination at concentrations as low as 20 ppm. As shown in FIG. 11 (right), excellent linearity of PS-spiked pork exudate sample analyzed by FAPCI-MS was obtained with calibration curve of y=1.86×10\hat{ }4 x−3.66×10\hat{ }4 (R²=0.99). FIG. 11 shows that a 20 ppm PS-spiked sample produced a signal (5.20E4) at m/z 207.12 approximately 21 times higher compared to the non-spiked sample (2.47E3). In this experiment, the measured PS concentration (22 ppm) closely matched the spiking value (20 ppm), with only an 10% discrepancy, confirming the accuracy and reliability of methods disclosed herein.

A commercially purchased salmon sample was further analyzed by FAPCI-MS after spiking with polystyrene (PS) nanoparticles. Briefly, 1.0 g of salmon meat was homogenized with 9 mL of deionized water, after which the homogenate was spiked with PS nanoparticles at defined concentrations and analyzed by FAPCI-MS. FIG. 12 (top left) shows a characteristic PS thermal decomposition ion at m/z 207.12 exhibited a signal intensity of 3.38×10⁵, approximately 130-fold higher than that observed for the non-spiked salmon sample (bottom left). Salmon samples spiked with varying concentrations of PS were subsequently analyzed to construct a calibration curve. As shown in FIG. 12c, excellent linearity was obtained with the regression equation y=3.36×10⁴x−2.39×10⁴ (R²=0.99). Triplicate analysis of the 100 ppm PS-spiked salmon sample yielded an average signal intensity of 2.78×10⁵, corresponding to a calculated PS concentration of 89.9 ppm based on the calibration curve. This represents a quantification deviation of approximately 10%, demonstrating that methods of the present disclosure can be used to quantify PS in complex biological matrices with a high degree of accuracy.

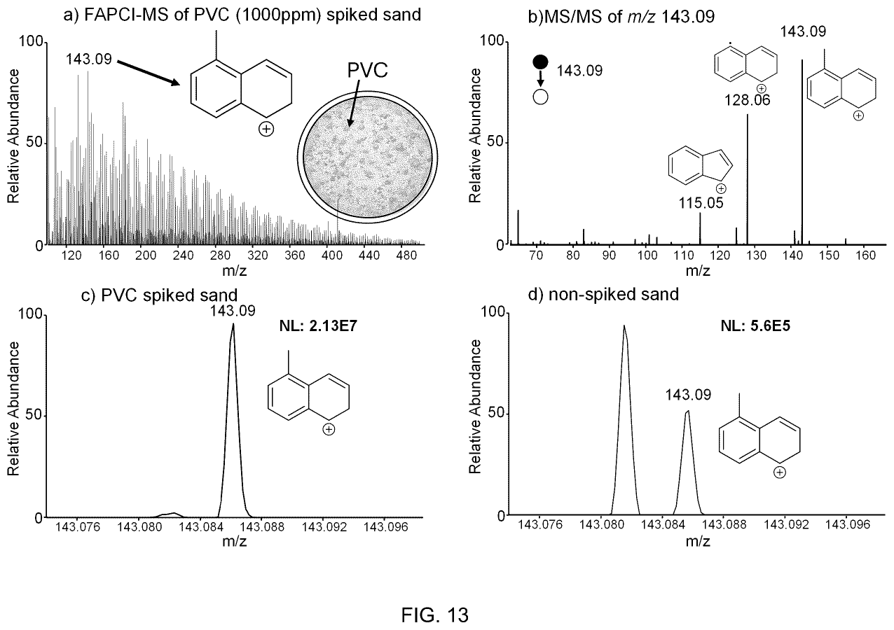

To test the PVC signal in a sample matrix, standard PVC microplastic (100-250 μm) was spiked into sand (purchased commercially) with 1000 ppm concentration and then analyzed by FAPCI-MS. FIG. 13 shows a MS spectrum (top left) obtained from FAPCI-MS analysis of a sand sample (ca. 1 mg) spiked with 1000 ppm-PVC, in which ion signals of PVC decomposition products, including aromatic hydrocarbons, conjugated polyenes, and hydrocarbons were seen with strong intensity, with the 1-methylnaphthalene ion (m/z 143.09) showing the strongest signal intensity. The structure of 1-methylnaphthalene was further confirmed by MS/MS analysis of m/z 143.09. FIG. 13 (top right) shows that upon collision-induced dissociation (CID), m/z 143.09 dissociated to yield m/z peaks at 115.05 and 128.06 from losses of C₂H₄, and CH₃, respectively. Comparing the signal intensity of 1-methylnaphthalene in PVC in spiked and non-spiked sand samples, the spiked sample showed signal intensity of 2.13×10⁷ for m/z 143.09 (FIG. 13, bottom left) 38-times greater than from the non-spiked sand sample (5.6×10⁵, FIG. 13, bottom right), demonstrating the feasibility and sensitivity of methods of the present disclosure for detecting PVC microplastics in sample matrices.

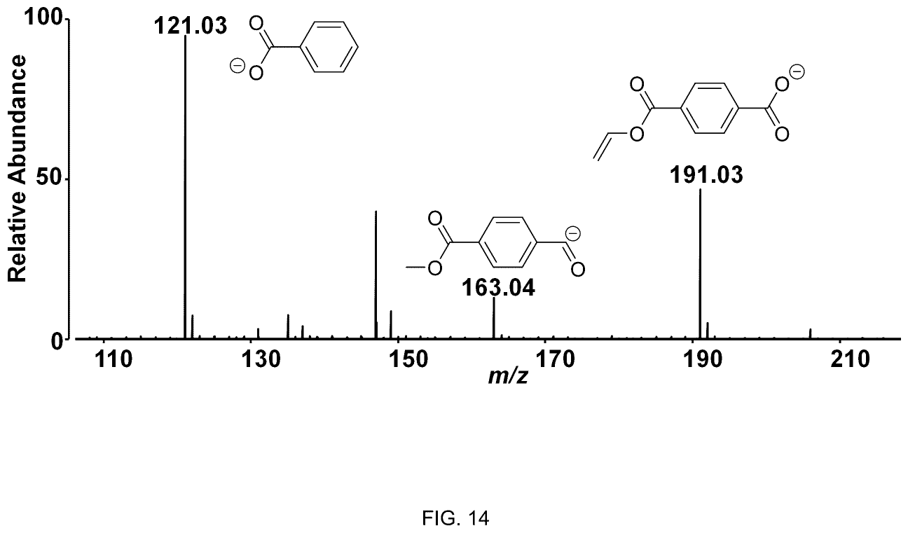

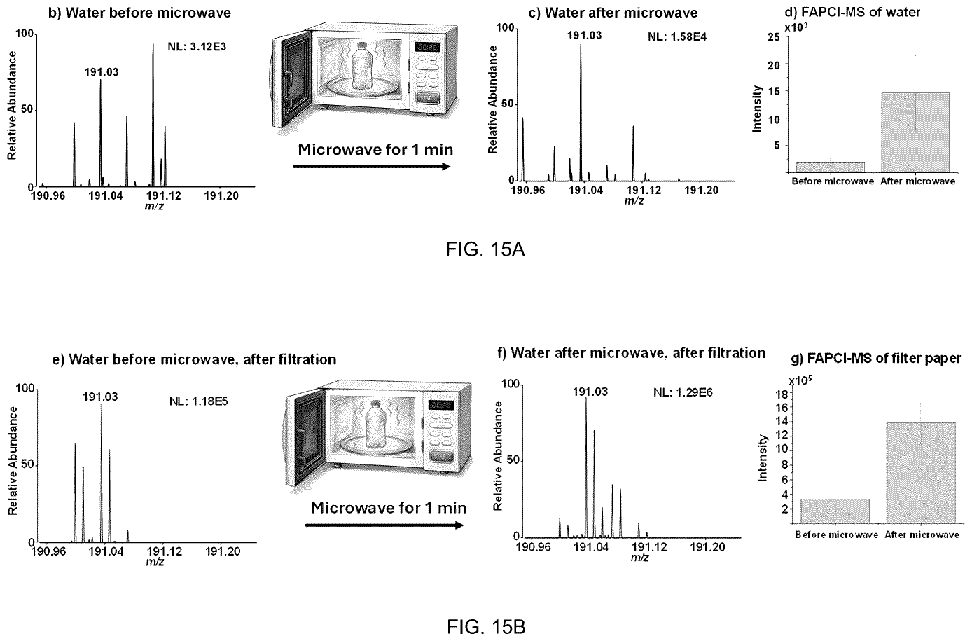

For PET detection of FAPCI-MS, standard PET microplastic was analyzed in negative mode with −3.5 kV voltage. FIG. 14 shows the spectrum obtained from MS analysis of standard PET (300 μm), with three characteristic negative ions at m/z 191.03 (vinyl terephthalate), m/z 163.04 (4-carbomethoxybenzoyl) and m/z 121.03 (benzoate). PET is the most widely used material for bottled water packaging, and PET microplastic contamination in bottled water has raised significant attention. Microwaved bottled water was investigated by methods of the present disclosure. In one example, water from commercially available PET plastic water bottles was directly microwaved under household conditions (1200 W) for 1 min, and PET particles levels compared in the microwaved water and non-microwaved water. For total PET particle analysis, 1 μL of water sample was directly placed on top of the sample plate (e.g., glass fiber cloth) and analyzed by FAPCI-MS. As shown in FIG. 15A, non-microwaved water exhibited a vinyl terephthalate (m/z 191.03) signal intensity of 3.12×10³, whereas the microwaved water showed an approximately five-fold higher intensity of 1.58×10⁴, while triplicate analysis results showed the same trend of increased PET ion signal after microwave heating. These results further demonstrate the robust ability of methods of the present disclosure to identify total PET particle content across size ranges.

To specifically evaluate PET MPs, size-fractionated PET MPs were collected using a two-step filtration procedure. Microwaved water samples (237 mL) were first filtered through a 2.5 μm pore-size filter (Whatman Grade 5, 43 mm diameter) to remove large PET particles, and the filtrate solution underwent a second filtration using a 0.45 μm pore-size glass fiber filter (OMICRON, 24 mm diameter). PET microplastics with sizes ranging from 0.45 μm to 2.5 μm were captured on the glass fiber filter. The filter was then cut into small pieces (3 mm²) and placed directly onto the sample plate for FAPCI-MS analysis. Control samples were prepared in the same manner using non-microwaved water. FIG. 15B shows spectra obtained from FAPCI-MS analysis of the filtered samples, in which vinyl terephthalate ion (m/z 191.03) was detected with signal intensities of 1.18×10⁵ from the non-microwaved sample and 1.29×10⁶ for the microwaved sample, wherein a 10-fold intensity increase for m/z 191.03 indicates that microwave heating does lead to increased number of PET microplastics (0.45-2.5 μm) released to the bottled water. Triplicate analysis (FIG. 15B, right) further confirmed the robustness and reproducibility of PET microplastic detection in microwaved bottled water by methods of the present disclosure. In comparison to traditional detection methods for MPs/NPs such as pyr-GC/MS, methods of the present disclosure offers several striking advantages. First, the present methods are rapid (e.g., as short as 10 second analysis time per sample) due to minimum sample preparation (filtration or simple drying), avoiding tedious sample extraction, digestion, or separation steps. Second, the present methods provide detailed molecular information about MPs and NPs due to inherent nature of MS detection, enabling differentiation among a variety of MP/NP species. Third, from analyzing ‘clean’ samples such as bottled water, to samples with complex matrices such as soil, methods of the present disclosure are tolerant to matrix effects and can be used for direct analysis of a wide range of sample times. Fourth, a reduced sample amount is required to perform analysis compared to existing methods (e.g., pyr-GC-MS).

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

### REFERENCES

ADDIN EN.REFLIST (1) Bajt, O. From plastics to microplastics and organisms. FEBS Open Bio 2021, 11 (4), 954-966. DOI: 10.1002/2211-5463.13120 From NLM.
(2) Möhlenkamp, P.; Purser, A.; Thomsen, L. Plastic microbeads from cosmetic products: an experimental study of their hydrodynamic behaviour, vertical transport and resuspension in phytoplankton and sediment aggregates. Elementa: Science of the Anthropocene 2018, 6. DOI: 10.1525/elementa.317 (acccessed Aug. 26, 2024).
(3) Mamun, A. A.; Prasetya, T. A. E.; Dewi, I. R.; Ahmad, M. Microplastics in human food chains: Food becoming a threat to health safety. Science of The Total Environment 2023, 858, 159834. DOI: https://doi.org/10.1016/j.scitotenv.2022.159834.
(4) Yee, M. S.; Hii, L. W.; Looi, C. K.; Lim, W. M.; Wong, S. F.; Kok, Y. Y.; Tan, B. K.; Wong, C. Y.; Leong, C. O. Impact of Microplastics and Nanoplastics on Human Health. Nanomaterials (Basel) 2021, 11 (2). DOI: 10.3390/nano11020496 From NLM.
(5) Ullah, S.; Ahmad, S.; Guo, X.; Ullah, S.; Ullah, S.; Nabi, G.; Wanghe, K. A review of the endocrine disrupting effects of micro and nano plastic and their associated chemicals in mammals. Front Endocrinol (Lausanne) 2022, 13, 1084236. DOI: 10.3389/fendo.2022.1084236.
(6) Qian, N.; Gao, X.; Lang, X.; Deng, H.; Bratu, T. M.; Chen, Q.; Stapleton, P.; Yan, B.; Min, W. Rapid single-particle chemical imaging of nanoplastics by SRS microscopy. Proceedings of the National Academy of Sciences 2024, 121 (3), e2300582121. DOI: doi: 10.1073/pnas.2300582121.
(7) Villegas-Camacho, O.; Alejo-Eleuterio, R.; Francisco-Valencia, I.; Granda-Gutiérrez, E.; Martínez-Gallegos, S.; Illescas, J. FTIR-Plastics: A Fourier Transform Infrared Spectroscopy dataset for the six most prevalent industrial plastic polymers. Data in Brief 2024, 55, 110612. DOI: https://doi.org/10.1016/j.dib.2024.110612.
(8) Trusler, M. M.; Sturrock, C. J.; Vane, C. H.; Cook, S.; Lomax, B. H. X-ray computed tomography: A novel non-invasive approach for the detection of microplastics in sediments? Marine Pollution Bulletin 2023, 194, 115350. DOI:
   https://doi.org/10.1016/j.marpolbul.2023.115350.
(9) Fabris, S.; Freire, M. T. d. A.; Wagner, R.; Reyes, F. G. A method to determine volatile contaminants in polyethylene terephthalate (PET) packages by HDC-GC-FID and its application to post-consumer materials. Food Science and Technology 2010, 30.
(10) Dhahak, A.; Grimmer, C.; Neumann, A.; Rüger, C.; Sklorz, M.; Streibel, T.; Zimmermann, R.; Mauviel, G.; Burkle-Vitzthum, V. Real time monitoring of slow pyrolysis of polyethylene terephthalate (PET) by different mass spectrometric techniques. Waste Management 2020, 106, 226-239. DOI: https://doi.org/10.1016/j.wasman.2020.03.028.
(11) Stauffer, E.; Dolan, J. A.; Newman, R. CHAPTER 5—Detection of Ignitable Liquid Residues at Fire Scenes. In Fire Debris Analysis, Stauffer, E., Dolan, J. A., Newman, R. Eds.; Academic Press, 2008; pp 131-161.
(12) Schofield, K. The enigmatic mechanism of the flame ionization detector: Its overlooked implications for fossil fuel combustion modeling. Progress in Energy and Combustion Science 2008, 34 (3), 330-350. DOI: https://doi.org/10.1016/j.pecs.2007.08.001.
(13) Law, C. K.; Sung, C. J. Structure, aerodynamics, and geometry of premixed flamelets. Progress in Energy and Combustion Science 2000, 26 (4), 459-505. DOI: https://doi.org/10.1016/S0360-1285 (00) 00018-6.
(14) Char, J.-M.; Yeh, J.-H. The measurement of open propane flame temperature using infrared technique. Journal of Quantitative Spectroscopy and Radiative Transfer 1996, 56 (1), 133-144.
(15) Albignac, M.; de Oliveira, T.; Landebrit, L.; Miquel, S.; Auguin, B.; Leroy, E.; Maria, E.; Mingotaud, A. F.; ter Halle, A. Tandem mass spectrometry enhances the performances of pyrolysis-gas chromatography-mass spectrometry for microplastic quantification. Journal of Analytical and Applied Pyrolysis 2023, 172, 105993. DOI: https://doi.org/10.1016/j.jaap.2023.105993.
(16) Brits, M.; van Poelgeest, B.; Nijenhuis, W.; van Velzen, M. J. M.; Béen, F. M.; Gruter, G. J. M.; Brandsma, S. H.; Lamoree, M. H. Quantitation of polystyrene by pyrolysis-GC-MS: The impact of polymer standards on micro and nanoplastic analysis. Polymer Testing 2024, 137, 108511. DOI: https://doi.org/10.1016/j.polymertesting.2024.108511.
(17) Zayoud, A.; Dao Thi, H.; Kusenberg, M.; Eschenbacher, A.; Kresovic, U.; Alderweireldt, N.; Djokic, M.; Van Geem, K. M. Pyrolysis of end-of-life polystyrene in a pilot-scale reactor: Maximizing styrene production. Waste Management 2022, 139, 85-95. DOI: https://doi.org/10.1016/j.wasman.2021.12.018.

What is claimed is:

1. A method for rapid analysis of plastic contaminants, comprising:
positioning a sample comprising a plastic contaminant a first distance from an inlet of a mass spectrometer;
heating the sample to an average temperature of from 200-2000° C. to produce thermal decomposition products of the plastic contaminant;
ionizing the thermal decomposition products; and
detecting ionized thermal decomposition products with the mass spectrometer.

2. The method of claim 1, wherein the plastic contaminant comprises a microplastic and/or nanoplastic.

3. The method of claim 1, wherein heating the sample comprises applying a flame, electric heat source, or laser impact.

4. The method of claim 3, wherein heating the sample comprises applying a flame directly or indirectly to the sample.

5. The method of claim 4, wherein the flame is generated from a fuel source selected from propane, butane, alcohol, or the like.

6. The method of claim 4, wherein applying the flame also ionizes the thermal decomposition products.

7. The method of claim 1, wherein ionizing the thermal decomposition products comprises using an ambient ionization method selected from flame ionization, atmospheric pressure chemical ionization, electrospray ionization, desorption electrospray ionization, sonic spray ionization, laser ionization, and plasma ionization.

8. The method of claim 1, further comprising preparing the sample from a medium containing a plastic contaminant.

9. The method of claim 8, wherein the medium is a powder, and preparing the sample comprises heating a metal element to redness and contacting the powder with the heated metal element to prepare an immobilized powder sample on the metal element.

10. The method of claim 8, wherein the medium is a liquid, and preparing the sample comprises passing the liquid through a filter to collect the plastic contaminant on the filter and drying the filter to prepare a dry filtrate sample.

11. The method of claim 8, wherein the medium is soil, and preparing the sample comprises drying an amount of the soil to remove moisture and placing the soil in a vial having an opening.

12. The method of claim 8, wherein the medium is a biological tissue and preparing the sample comprises homogenizing the biological tissue and depositing an amount of the homogenized tissue on a substrate.

13. The method of claim 1, wherein the method is completed in 10 seconds or less.

14. The method of claim 3, wherein the sample is on a vial, substrate, or support, and heating the sample comprises heating the sample indirectly by heating the vial, substrate or support.

15. The method of claim 14, wherein ionizing the thermal decomposition products comprises applying an ambient ionization method above the sample while heating the sample indirectly, the ambient ionization method selected from flame ionization, atmospheric pressure chemical ionization, electrospray ionization, desorption electrospray ionization, sonic spray ionization, laser ionization, and plasma ionization.

16. The method of claim 14, wherein ionizing the thermal decomposition products comprises applying a propane flame having an average temperature of from 500-2000° C. above the sample while heating the sample indirectly.

17. The method of claim 14, wherein ionizing the thermal decomposition products comprises applying a voltage in a range of from 1 to 5.0 kV to a metal needle placed above the sample while heating the sample indirectly.

18. The method of claim 14, wherein the vial, substrate or support comprises glass, glass fiber, or metal.

19. The method of claim 1, wherein the ionized thermal decomposition products comprise one or more characteristic ions of the plastic contaminant.

20. The method of claim 19, wherein the plastic contaminant comprises polyethylene terephthalate (PET), and the characteristic ions comprise one or more of vinyl terephthalate cation (m/z about 193.05) and 4-acetylbenzaldehyde cation (m/z about 149.06) with the mass spectrometer in positive ion mode, or vinyl terephthalate anion (m/z about 191.03) with the mass spectrometer in negative ion mode.

21. The method of claim 19, wherein the plastic contaminant comprises polystyrene (PS) and the characteristic ions comprise one or more of 1,3-diphenylpropene (m/z about 195.116) and but-3-ene-1,3-diphenylpropene (m/z about 207.116) with the mass spectrometer in positive ion mode.

22. The method of claim 1, further comprising quantifying an amount of the plastic contaminant present in the sample by analyzing a signal intensity of a detected ionized thermal decomposition product.

23. The method of claim 1, further comprising analyzing one or more detected ionized thermal decomposition products using tandem mass spectrometry (MS/MS).
